SEQUENCE LISTING (3 sequences)

SEQ ID NO: 1 - DNA, 15 bp, Artificial Sequence
  Description of Artificial Sequence Synthetic oligonucleotide
aactgactaacttga

SEQ ID NO: 2 - DNA, 15 bp, Artificial Sequence
  Description of Artificial Sequence Synthetic oligonucleotide
aactgactaatttga

SEQ ID NO: 3 - DNA, 34 bp, Artificial Sequence
  Description of Artificial Sequence Synthetic oligonucleotide
cgattatactatgcatgccatcgattattacaat

(19) United States
(12) Patent Application Publication
(10) Pub. No.: US 2023/0103457 A1
(43) Pub. Date: Apr. 6, 2023

(54) METHOD FOR IDENTIFYING VARIANTS IN GENE PRODUCTS FROM GENE CONSTRUCTS USED IN CELL THERAPY APPLICATIONS

(71) Applicant: Kite Pharma, Inc., Santa Monica, CA (US)

(72) Inventors: Jorge Andrade, Las Vegas, NV (US); Eugene Bolotin, Berkeley, CA (US); Alexander S. Falk, Thousand Oaks, CA (US); Edward H. Liao, Petaluma, CA (US)

(21) Appl. No.: 17/853,708

(22) Filed: Jun. 29, 2022

Related U.S. Application Data
(60) Provisional application No. 63/217,933, filed on Jul. 2, 2021.

(51) Int. Cl. G16B 20/20; C12Q 1/6869

(52) U.S. Cl. CPC G16B 20/20; C12Q 1/6869

(57) ABSTRACT

A method for ensuring that gene products used in cell therapy do not carry a risk of reduced efficacy or toxicity due to production of unintended variants. The method includes performing an in-silico analysis on the gene construct to identify and alter sequences likely to cause variants. Also, the method includes performing an in-vivo analysis consisting of RNA-sequence of construct based products. Variant detection may then be performed based on gapped reads from the RNA-sequence to determine variant expression levels, variant significance. The method may include repeating the in-silico analysis if identified variants are unacceptable.

### CROSS REFERENCE TO RELATED APPLICATIONS

This application claims priority to U.S. Provisional Patent Application No. 63/217,933 filed on Jul. 2, 2021, the entire contents of which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Nov. 30, 2022, is named K-1132-US-NP_SL.txt and is 1,045 bytes in size.

### FIELD

The disclosure relates to methods for detecting and replacing a sequence which may cause an undesired variant in a gene construct.

### BACKGROUND

In recent years, advances in medical technology have led to the emerging use of immunotherapies to treat different types of illnesses and diseases, including various forms of cancer. Generally, immunotherapy is the treatment of disease by stimulating or suppressing an immune response. Often, modified versions of a patient's own biological material, such as immune cells, are reintroduced into the patient in order to initiate and/or supplement the immune response.

For example, engineered immune cells have been shown to possess desired qualities in therapeutic treatments, particularly in oncology. Two main types of engineered immune cells are those that contain chimeric antigen receptors (termed “CARs” or “CAR-Ts”) and T-cell receptors (“TCRs”). These engineered cells are engineered to endow them with antigen specificity while retaining or enhancing their ability to recognize and kill a target cell. Chimeric antigen receptors may comprise, for example, (i) an antigen-specific component (“antigen binding molecule”), (ii) an extracellular domain, (iii) one or more costimulatory domains, and (iv) one or more activating domains. Each domain may be heterogeneous, that is, comprised of sequences derived from (or corresponding to) different protein chains.

Introduction of genetic elements into cells through the use of gene constructs (such as viral vectors) is one method to produce cells for applications such as cell therapy. Gene construct production and transduction of cells requires multiple biological steps that have the potential to introduce heterogeneity into a product. With specific request to viral vectors, defects in viral packaging, transduction, or transgene transcription, can introduce undesirable contaminants that differ from the intended sequence. These “variants” may express unexpected protein sequences and, depending on their frequency and nature, may reduce efficacy, compromise manufacturing or even increase side effects such as toxicity. It is critical to reduce the potential for variant production during the transgene development stage and during cell therapy development in order to prevent possible program failures.

What is needed is a systematic method for detection and identification of variants and potential variant causing sequences in gene products used in cell therapy applications. Also, what is needed is an improved method for de-risking strategies such as variant characterization and/or DNA sequence modifications to remove detected variants can be employed in an iterative process if variants are identified.

### SUMMARY

Briefly, and in general terms, the present disclosure is directed to a system and method for creating a gene product used in cell therapy. In one embodiment, the method includes performing an in silico analysis on the gene construct to identify and alter sequences that cause variants. Also, the method includes performing an in vivo analysis including a first RNA-sequencing step to identify a frequency percentage of variants and repeating the in silico analysis if the first RNA-sequencing step identifies greater than 5% frequency of variants in the gene construct. The in vivo analysis may include a second RNA-sequencing step to identify a frequency percentage of variants and repeating the in silico analysis if variants are not acceptable. The method may also include performing variant detection based on gapped reads from at least one of the first and second RNA-sequencing to determine variant expression levels and variant significance. The in silico analysis may be repeated to create a new gene construct if the variant is determined to be unacceptable.

In one embodiment of the disclosed method, the in silico analysis may include modifying the variant in the gene construct with synonymous codon substitution. Furthermore, the in silico analysis may include identifying and removing a homologous sequence from the gene construct. In one embodiment, the in silico analysis includes identifying identical sequences in the gene construct. In addition, the in silico analysis may include calculating a matrix of subsection combinations from the input sequence and acquiring a Hamming distance for each combination. The method of one embodiment includes substituting random synonymous codons if the substitutions increase the sum over the matrix.

In one embodiment, the in vivo analysis includes RNA-sequencing of the products made from the gene construct. In one embodiment, the RNA-sequencing is performed in multiple stages to identify high and then low frequency variants. Furthermore, the in vivo analysis may include conducting analysis to determine if the lower frequency variant should be replaced.

In one embodiment of the method, the variant detection includes extracting RNA from a donor sample. Also, when performing gap aware alignment, three separate aligners may be used in one embodiment. The P values for variant significance are calculated using the Wilcox Rank Sum Test.

The present disclosure is also directed to a method for ensuring that gene products used in cell therapy do not carry a risk of reduced efficacy or toxicity due to production of unintended variants. The method includes performing an in-silico analysis on the gene construct to identify and alter sequences likely to cause variants. Also, the method includes performing an in-vivo analysis consisting of RNA-sequence of construct-based products. Variant detection may then be performed based on gapped reads from the RNA-sequence to determine variant expression levels, variant significance. The method may include repeating the in-silico analysis if identified variants are unacceptable.

An embodiment of the disclosure relates to a method for detecting and replacing a sequence which may cause an undesired variant in a gene construct. Such a method includes: performing an in-silico analysis of the gene construct to detect a presence of the sequence which may cause the undesired variant; replacing the detected sequence which may cause the undesired variant with an alternative sequence, where the alternative sequence is derived comprising synonymous codon substitution; measuring a frequency percentage of the undesired variant expressed by the gene construct comprising performing an in-vivo analysis of one or more genes expressed by the gene construct comprising performing a RNA-sequencing analysis of an RNA product transcribed from the gene construct, where the frequency percentage of the undesired variant is determined at least in part by using a splice-aware aligner from the RNA-sequencing analysis; and repeating the in-silico analysis and replacing steps if the frequency percentage of the undesired variant in the gene product from the in-vivo analysis is greater than a predetermined value of acceptable frequency percentage of the undesired variant.

An embodiment of the disclosure relates to a method for creating a gene product used in cell therapy. Such a method includes the steps of: performing an in-silico analysis on a gene construct encoding the gene product to identify and alter a sequence that may causes an undesired variant; replacing the detected sequence which may cause the undesired variant with an alternative sequence, where the alternative sequence is derived comprising synonymous codon substitution; measuring a frequency percentage of the undesired variant expressed by the gene construct comprising performing an in-vivo analysis of one or more genes expressed by the gene construct comprising performing a RNA-sequencing analysis of an RNA product transcribed from the gene construct, where the frequency percentage of the undesired variant is determined at least in part by using a splice-aware aligner from the RNA-sequencing analysis; repeating the in-silico and replacing steps to create a new gene construct if the frequency percentage of the undesired variant in the gene product from the in-vivo analysis is greater than a predetermined value of acceptable frequency percentage of the undesired variant; and measuring a frequency percentage of the undesired variant expressed by the new gene construct comprising performing an in-vivo analysis of one or more genes expressed by the new gene construct comprising performing a RNA-sequencing analysis of an RNA product transcribed from the new gene construct, where the frequency percentage of the undesired variant is determined at least in part by using a splice-aware aligner from the RNA-sequencing analysis.

Other aspects and advantages of the technology will become apparent from the following detailed description, taken in conjunction with the accompanying drawings, illustrating the principles of the technology by way of example only.

### BRIEF DESCRIPTION OF THE DRAWINGS

The teachings claimed and/or described herein are further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 depicts an overview of one possibility for created a variant during cell therapy manufacturing.

FIG. 2 depicts one embodiment of a variant prediction, detection and elimination process.

FIG. 3 depicts an example of a Sequence Diverger matrix that may be used in the disclosed process.

FIG. 4 depicts an exemplary process of a repeat remover tool that may be used in the disclosed process. Figure discloses SEQ ID NOS 1, 2, and 2, respectively, in order of appearance.

FIG. 5 depicts an example of a screen shot showing a variant report template that may be used in the disclosed process.

FIG. 6 depicts an example of a screen shot showing the output from a Repeat Finder and Visualizer tool that identifies identical sequences. Figure discloses SEQ ID NO: 3.

FIG. 7A is a flow diagram depicting an exemplary process for detecting and replacing a sequence which may cause an undesired variant in a gene construct, according to an embodiment of the disclosure.

FIG. 7B is a flow diagram depicting an exemplary process for creating a gene therapy product used in cell therapy, according to an embodiment of the disclosure.

FIGS. 8A-8C depict example code portions that may be used to practice the disclosed methods.

### DETAILED DESCRIPTION

The present disclosure addresses the need for an improved system and method to identify variants in gene constructs and then selecting a gene construct for use in cell therapy. The below disclosure describes a systematic method for detection and identification of variants and potential variant causing sequences in gene products used in cell therapy applications. When variants are identified, de-risking strategies such as variant characterization and/or DNA sequence modifications to remove detected variants can be employed in an iterative process.

It will be understood that descriptions herein are exemplary and explanatory only and are not restrictive of the technology as claimed. In this application, the use of the singular includes the plural unless specifically stated otherwise.

All documents, or portions of documents, cited in this application, including but not limited to patents, patent applications, articles, books, and treatises, are hereby expressly incorporated by reference in their entirety for any purpose. As utilized in accordance with the present disclosure, the following terms, unless otherwise indicated, shall be understood to have the following meanings:

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”.

The term “and/or” where used herein is to be taken as specific disclosure of each of the two specified features or components with or without the other. Thus, the term “and/or” as used in a phrase such as “A and/or B” herein is intended to include A and B; A or B; A (alone); and B (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “e.g.,” and “i.e.” as used herein, are used merely by way of example, without limitation intended, and should not be construed as referring only those items explicitly enumerated in the specification.

The terms “or more”, “at least”, “more than”, and the like, e.g., “at least one” are understood to include but not be limited to at least 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more than the stated value. Also included is any greater number or fraction in between.

Conversely, the term “no more than” includes each value less than the stated value. For example, “no more than 100 nucleotides” includes 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, and 0 nucleotides. Also included is any lesser number or fraction in between.

The terms “plurality”, “at least two”, “two or more”, “at least second”, and the like, are understood to include but not limited to at least 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more. Also included is any greater number or fraction in between.

Unless specifically stated or evident from context, as used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “approximately” may mean within one or more than one standard deviation per the practice in the art. “About” or “approximately” may mean a range of up to 10% (i.e., ±10%). Thus, “about” may be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg may include any amount between 4.5 mg and 5.5 mg. Furthermore, particularly with respect to biological systems or processes, the terms may mean up to an order of magnitude or up to 5-fold of a value. When particular values or compositions are provided in the instant disclosure, unless otherwise stated, the meaning of “about” or “approximately” should be assumed to be within an acceptable error range for that particular value or composition.

As described herein, any concentration range, percentage range, ratio range or integer range is to be understood to be inclusive of the value of any integer within the recited range and, when appropriate, fractions thereof (such as one-tenth and one-hundredth of an integer), unless otherwise indicated.

Units, prefixes, and symbols used herein are provided using their Système International de Unites (SI) accepted form. Numeric ranges are inclusive of the numbers defining the range.

Unless defined otherwise, all technical and scientific terms used herein have the same meaning as commonly understood by one of ordinary skill in the art to which this disclosure is related. For example, Juo, “The Concise Dictionary of Biomedicine and Molecular Biology”, 2nd ed., (2001), CRC Press; “The Dictionary of Cell & Molecular Biology”, 5th ed., (2013), Academic Press; and “The Oxford Dictionary Of Biochemistry And Molecular Biology”, Cammack et al. eds., 2nd ed, (2006), Oxford University Press, provide those of skill in the art with a general dictionary for many of the terms used in this disclosure.

“Administering” refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. Exemplary routes of administration for the compositions disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, e.g., orally. Other non-parenteral routes include a topical, epidermal or mucosal route of administration, for example, intranasally, vaginally, rectally, sublingually or topically. Administering may also be performed, for example, once, a plurality of times, and/or over one or more extended periods. In one embodiment, the CAR T cell treatment is administered via an “infusion product” comprising CAR T cells.

The term “antibody” (Ab) includes, without limitation, a glycoprotein immunoglobulin which binds specifically to an antigen. In general, an antibody may comprise at least two heavy (H) chains and two light (L) chains interconnected by disulfide bonds, or an antigen-binding molecule thereof. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region comprises one constant domain, CL. The VH and VL regions may be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component (C1q) of the classical complement system.

Antibodies may include, for example, monoclonal antibodies, recombinantly produced antibodies, monospecific antibodies, multispecific antibodies (including bispecific antibodies), human antibodies, engineered antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, antibody fusions (sometimes referred to herein as “antibody conjugates”), heteroconjugate antibodies, single domain antibodies, monovalent antibodies, single chain antibodies or single-chain Fvs (scFv), camelized antibodies, affybodies, Fab fragments, F(ab′)2 fragments, disulfide-linked Fvs (sdFv), anti-idiotypic (anti-Id) antibodies (including, e.g., anti-anti-Id antibodies), minibodies, domain antibodies, synthetic antibodies (sometimes referred to herein as “antibody mimetics”), and antigen-binding fragments of any of the above. In some embodiments, antibodies described herein refer to polyclonal antibody populations.

An “antigen binding molecule,” “antigen binding portion,” or “antibody fragment” refers to any molecule that comprises the antigen binding parts (e.g., CDRs) of the antibody from which the molecule is derived. An antigen binding molecule may include the antigenic complementarity determining regions (CDRs). Examples of antibody fragments include, but are not limited to, Fab, Fab′, F(ab′)2, and Fv fragments, dAb, linear antibodies, scFv antibodies, and multispecific antibodies formed from antigen binding molecules. Peptibodies (i.e., Fc fusion molecules comprising peptide binding domains) are another example of suitable antigen binding molecules. In some embodiments, the antigen binding molecule binds to an antigen on a tumor cell. In some embodiments, the antigen binding molecule binds to an antigen on a cell involved in a hyperproliferative disease or to a viral or bacterial antigen. In some embodiments, the antigen binding molecule binds to CD19. In further embodiments, the antigen binding molecule is an antibody fragment that specifically binds to the antigen, including one or more of the complementarity determining regions (CDRs) thereof. In further embodiments, the antigen binding molecule is a single chain variable fragment (scFv). In some embodiments, the antigen binding molecule comprises or consists of avimers.

An “antigen” refers to any molecule that provokes an immune response or is capable of being bound by an antibody or an antigen binding molecule. The immune response may involve either antibody production, or the activation of specific immunologically-competent cells, or both. A person of skill in the art would readily understand that any macromolecule, including virtually all proteins or peptides, may serve as an antigen. An antigen may be endogenously expressed, i.e. expressed by genomic DNA, or may be recombinantly expressed. An antigen may be specific to a certain tissue, such as a cancer cell, or it may be broadly expressed. In addition, fragments of larger molecules may act as antigens. In some embodiments, antigens are tumor antigens.

The term “neutralizing” refers to an antigen binding molecule, scFv, antibody, or a fragment thereof, that binds to a ligand and prevents or reduces the biological effect of that ligand. In some embodiments, the antigen binding molecule, scFv, antibody, or a fragment thereof, directly blocks a binding site on the ligand or otherwise alters the ligand's ability to bind through indirect means (such as structural or energetic alterations in the ligand). In some embodiments, the antigen binding molecule, scFv, antibody, or a fragment thereof prevents the protein to which it is bound from performing a biological function.

The term “autologous” refers to any material derived from the same individual to which it is later to be re-introduced. For example, the engineered autologous cell therapy (eACT™) method described herein involves collection of lymphocytes from a patient, which are then engineered to express, e.g., a CAR construct, and then administered back to the same patient.

The term “allogeneic” refers to any material derived from one individual which is then introduced to another individual of the same species, e.g., allogeneic T cell transplantation.

The terms “transduction” and “transduced” refer to the process whereby foreign DNA is introduced into a cell via viral vector (see Jones et al., “Genetics: principles and analysis,” Boston: Jones & Bartlett Publ. (1998)). In some embodiments, the vector is a retroviral vector, a DNA vector, a RNA vector, an adenoviral vector, a baculoviral vector, an Epstein Barr viral vector, a papovaviral vector, a vaccinia viral vector, a herpes simplex viral vector, an adenovirus associated vector, a lentiviral vector, or any combination thereof.

A “cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” may include a tumor. In this application, the term cancer is synonymous with malignancy. Examples of cancers that may be treated by the methods disclosed herein include, but are not limited to, cancers of the immune system including lymphoma, leukemia, myeloma, and other leukocyte malignancies. In some embodiments, the methods disclosed herein may be used to reduce the tumor size of a tumor derived from, for example, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, [add other solid tumors] multiple myeloma, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia, chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T cell lymphoma, environmentally induced cancers including those induced by asbestos, other B cell malignancies, and combinations of said cancers. In some embodiments, the cancer is multiple myeloma. In some embodiments, the cancer is NHL. The particular cancer may be responsive to chemo- or radiation therapy or the cancer may be refractory. A refractory cancer refers to a cancer that is not amenable to surgical intervention and the cancer is either initially unresponsive to chemo- or radiation therapy or the cancer becomes unresponsive over time.

An “anti-tumor effect” as used herein, refers to a biological effect that may present as a decrease in tumor volume, a decrease in the number of tumor cells, a decrease in tumor cell proliferation, a decrease in the number of metastases, an increase in overall or progression-free survival, an increase in life expectancy, or amelioration of various physiological symptoms associated with the tumor. An anti-tumor effect may also refer to the prevention of the occurrence of a tumor, e.g., a vaccine.

A “cytokine,” as used herein, refers to a non-antibody protein that is released by one cell in response to contact with a specific antigen, wherein the cytokine interacts with a second cell to mediate a response in the second cell. “Cytokine” as used herein is meant to refer to proteins released by one cell population that act on another cell as intercellular mediators. A cytokine may be endogenously expressed by a cell or administered to a subject. Cytokines may be released by immune cells, including macrophages, B cells, T cells, and mast cells to propagate an immune response. Cytokines may induce various responses in the recipient cell. Cytokines may include homeostatic cytokines, chemokines, pro-inflammatory cytokines, effectors, and acute-phase proteins. For example, homeostatic cytokines, including interleukin (IL) 7 and IL-15, promote immune cell survival and proliferation, and pro-inflammatory cytokines may promote an inflammatory response. Examples of homeostatic cytokines include, but are not limited to, IL-2, IL-4, IL-5, IL-7, IL-10, IL-12p40, IL-12p70, IL-15, and interferon (IFN) gamma Examples of pro-inflammatory cytokines include, but are not limited to, IL-1a, IL-1b, IL-6, IL-13, IL-17a, tumor necrosis factor (TNF)-alpha, TNF-beta, fibroblast growth factor (FGF) 2, granulocyte macrophage colony-stimulating factor (GM-CSF), soluble intercellular adhesion molecule 1 (sICAM-1), soluble vascular adhesion molecule 1 (sVCAM-1), vascular endothelial growth factor (VEGF), VEGF-C, VEGF-D, and placental growth factor (PLGF). Examples of effectors include, but are not limited to, granzyme A, granzyme B, soluble Fas ligand (sFasL), and perforin. Examples of acute phase-proteins include, but are not limited to, C-reactive protein (CRP) and serum amyloid A (SAA).

“Chemokines” are a type of cytokine that mediates cell chemotaxis, or directional movement. Examples of chemokines include, but are not limited to, IL-8, IL-16, eotaxin, eotaxin-3, macrophage-derived chemokine (MDC or CCL22), monocyte chemotactic protein 1 (MCP-1 or CCL2), MCP-4, macrophage inflammatory protein 1α (MIP-1α, MIP-1α), MIP-1β (MIP-1b), gamma-induced protein 10 (IP-10), and thymus and activation regulated chemokine (TARC or CCL17).

As used herein, “chimeric receptor” refers to an engineered surface expressed molecule capable of recognizing a particular molecule. Chimeric antigen receptors (CARs) and engineered T cell receptors (TCRs), which comprise binding domains capable of interacting with a particular tumor antigen, allow T cells to target and kill cancer cells that express the particular tumor antigen. In one embodiment, the T cell treatment is based on T cells engineered to express a chimeric antigen receptor (CAR) or a T cell receptor (TCR), which comprises (i) an antigen binding molecule, (ii) a costimulatory domain, and (iii) an activating domain. The costimulatory domain may comprise an extracellular domain, a transmembrane domain, and an intracellular domain, wherein the extracellular domain comprises a hinge domain, which may be truncated.

A “therapeutically effective amount,” “effective dose,” “effective amount,” or “therapeutically effective dosage” of a therapeutic agent, e.g., engineered CAR T cells, is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. Such terms can be used interchangeably. The ability of a therapeutic agent to promote disease regression may be evaluated using a variety of methods known to the skilled practitioner, such as in human subjects during clinical trials, in animal model systems predictive of efficacy in humans, or by assaying the activity of the agent in in vitro assays.

The term “lymphocyte” as used herein includes natural killer (NK) cells, T cells, or B cells. NK cells are a type of cytotoxic (cell toxic) lymphocyte that represent a major component of the inherent immune system. NK cells reject tumors and cells infected by viruses. It works through the process of apoptosis or programmed cell death. They were termed “natural killers” because they do not require activation in order to kill cells. T cells play a major role in cell-mediated-immunity (no antibody involvement). Its T cell receptors (TCR) differentiate themselves from other lymphocyte types. The thymus, a specialized organ of the immune system, is primarily responsible for the T cell's maturation. There are six types of T cells, namely: Helper T cells (e.g., CD4+ cells), Cytotoxic T cells (also known as TC, cytotoxic T lymphocyte, CTL, T-killer cell, cytolytic T cell, CD8+ T cells or killer T cell), Memory T cells ((i) stem memory TSCM cells, like naive cells, are CD45RO−, CCR7+, CD45RA+, CD62L+ (L-selectin), CD27+, CD28+ and IL-7Rα+, but they also express large amounts of CD95, IL-2Rβ, CXCR3, and LFA-1, and show numerous functional attributes distinctive of memory cells); (ii) central memory TCM cells express L-selectin and the CCR7, they secrete IL-2, but not IFNγ or IL-4, and (iii) effector memory TEM cells, however, do not express L-selectin or CCR7 but produce effector cytokines like IFNγ and IL-4), Regulatory T cells (Tregs, suppressor T cells, or CD4+CD25+ regulatory T cells), Natural Killer T cells (NKT) and Gamma Delta T cells. B-cells, on the other hand, play a principal role in humoral immunity (with antibody involvement). It makes antibodies and antigens and performs the role of antigen-presenting cells (APCs) and turns into memory B-cells after activation by antigen interaction. In mammals, immature B-cells are formed in the bone marrow, where its name is derived from.

The term “genetically engineered” or “engineered” refers to a method of modifying the genome of a cell, including, but not limited to, deleting a coding or non-coding region or a portion thereof or inserting a coding region or a portion thereof. In some embodiments, the cell that is modified is a lymphocyte, e.g., a T cell, which may either be obtained from a patient or a donor. The cell may be modified to express an exogenous construct, such as, e.g., a chimeric antigen receptor (CAR) or a T cell receptor (TCR), which is incorporated into the cell's genome.

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including Abs, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

The term “immunotherapy” refers to the treatment of a subject afflicted with, or at risk of contracting or suffering a recurrence of, a disease by a method comprising inducing, enhancing, suppressing or otherwise modifying an immune response. Examples of immunotherapy include, but are not limited to, T cell therapies. T cell therapy may include adoptive T cell therapy, tumor-infiltrating lymphocyte (TIL) immunotherapy, autologous cell therapy, engineered autologous cell therapy (eACT™), and allogeneic T cell transplantation. However, one of skill in the art would recognize that the conditioning methods disclosed herein would enhance the effectiveness of any transplanted T cell therapy. Examples of T cell therapies are described in U.S. Patent Publication Nos. 2014/0154228 and 2002/0006409, U.S. Pat. Nos. 7,741,465, 6,319,494, 5,728,388, and International Publication No. WO 2008/081035. In some embodiments, the immunotherapy comprises CAR T cell treatment. In some embodiments, the CAR T cell treatment product is administered via infusion.

The T cells of the immunotherapy may come from any source known in the art. For example, T cells may be differentiated in vitro from a hematopoietic stem cell population, or T cells may be obtained from a subject. T cells may be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the T cells may be derived from one or more T cell lines available in the art. T cells may also be obtained from a unit of blood collected from a subject using any number of techniques known to the skilled artisan, such as FICOLL™ separation and/or apheresis. Additional methods of isolating T cells for a T cell therapy are disclosed in U.S. Patent Publication No. 2013/0287748, which is herein incorporated by reference in its entirety.

The term “engineered Autologous Cell Therapy,” or “eACT™,” also known as adoptive cell transfer, is a process by which a patient's own T cells are collected and subsequently genetically altered to recognize and target one or more antigens expressed on the cell surface of one or more specific tumor cells or malignancies. T cells may be engineered to express, for example, chimeric antigen receptors (CAR). CAR positive (+) T cells are engineered to express an extracellular single chain variable fragment (scFv) with specificity for a particular tumor antigen linked to an intracellular signaling part comprising at least one costimulatory domain and at least one activating domain. The CAR scFv may be designed to target, for example, CD19, which is a transmembrane protein expressed by cells in the B cell lineage, including all normal B cells and B cell malignances, including but not limited to diffuse large B-cell lymphoma (DLBCL) not otherwise specified, primary mediastinal large B-cell lymphoma, high grade B-cell lymphoma, and DLBCL arising from follicular lymphoma, NHL, CLL, and non-T cell ALL. Example CAR T cell therapies and constructs are described in U.S. Patent Publication Nos. 2013/0287748, 2014/0227237, 2014/0099309, and 2014/0050708, and these references are incorporated by reference in their entirety.

A “patient” as used herein includes any human who is afflicted with a cancer (e.g., a lymphoma or a leukemia). The terms “subject” and “patient” are used interchangeably herein.

As used herein, the term “in vitro cell” refers to any cell which is cultured ex vivo. In particular, an in vitro cell may include a T cell. The term “in vivo” means within the patient.

The terms “peptide,” “polypeptide,” and “protein” are used interchangeably, and refer to a compound comprised of amino acid residues covalently linked by peptide bonds. A protein or peptide contains at least two amino acids, and no limitation is placed on the maximum number of amino acids that may comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

“Stimulation,” as used herein, refers to a primary response induced by binding of a stimulatory molecule with its cognate ligand, wherein the binding mediates a signal transduction event. A “stimulatory molecule” is a molecule on a T cell, e.g., the T cell receptor (TCR)/CD3 complex that specifically binds with a cognate stimulatory ligand present on an antigen present cell. A “stimulatory ligand” is a ligand that when present on an antigen presenting cell (e.g., an APC, a dendritic cell, a B-cell, and the like) may specifically bind with a stimulatory molecule on a T cell, thereby mediating a primary response by the T cell, including, but not limited to, activation, initiation of an immune response, proliferation, and the like. Stimulatory ligands include, but are not limited to, an anti-CD3 antibody, an MHC Class I molecule loaded with a peptide, a superagonist anti-CD2 antibody, and a superagonist anti-CD28 antibody.

A “costimulatory signal,” as used herein, refers to a signal, which in combination with a primary signal, such as TCR/CD3 ligation, leads to a T cell response, such as, but not limited to, proliferation and/or upregulation or down regulation of key molecules.

A “costimulatory ligand,” as used herein, includes a molecule on an antigen presenting cell that specifically binds a cognate co-stimulatory molecule on a T cell. Binding of the costimulatory ligand provides a signal that mediates a T cell response, including, but not limited to, proliferation, activation, differentiation, and the like. A costimulatory ligand induces a signal that is in addition to the primary signal provided by a stimulatory molecule, for instance, by binding of a T cell receptor (TCR)/CD3 complex with a major histocompatibility complex (MHC) molecule loaded with peptide. A co-stimulatory ligand may include, but is not limited to, 3/TR6, 4-1BB ligand, agonist or antibody that binds Toll ligand receptor, B7-1 (CD80), B7-2 (CD86), CD30 ligand, CD40, CD7, CD70, CD83, herpes virus entry mediator (HVEM), human leukocyte antigen G (HLA-G), ILT4, immunoglobulin-like transcript (ILT) 3, inducible costimulatory ligand (ICOS-L), intercellular adhesion molecule (ICAM), ligand that specifically binds with B7-H3, lymphotoxin beta receptor, MHC class I chain-related protein A (MICA), MHC class I chain-related protein B (MICB), OX40 ligand, PD-L2, or programmed death (PD) L1. In certain embodiments, a co-stimulatory ligand includes, without limitation, an antibody that specifically binds with a co-stimulatory molecule present on a T cell, such as, but not limited to, 4-1BB, B7-H3, CD2, CD27, CD28, CD30, CD40, CD7, ICOS, ligand that specifically binds with CD83, lymphocyte function-associated antigen-1 (LFA-1), natural killer cell receptor C (NKG2C), OX40, PD-1, or tumor necrosis factor superfamily member 14 (TNFSF14 or LIGHT).

A “costimulatory molecule” is a cognate binding partner on a T cell that specifically binds with a costimulatory ligand, thereby mediating a costimulatory response by the T cell, such as, but not limited to, proliferation. Costimulatory molecules include, but are not limited to, 4-1BB/CD137, B7-H3, BAFFR, BLAME (SLAMF8), BTLA, CD33, CD45, CD100 (SEMA4D), CD103, CD134, CD137, CD154, CD16, CD160 (BY55), CD18, CD19, CD19a, CD2, CD22, CD247, CD27, CD276 (B7-H3), CD28, CD29, CD3 (alpha; beta; delta; epsilon; gamma; zeta), CD30, CD37, CD4, CD4, CD40, CD49a, CD49D, CD49f, CD5, CD64, CD69, CD7, CD80, CD83 ligand, CD84, CD86, CD8alpha, CD8beta, CD9, CD96 (Tactile), CD11a, CD11b, CD11c, CD11d, CDS, CEACAM1, CRT AM, DAP-10, DNAM1 (CD226), Fc gamma receptor, GADS, GITR, HVEM (LIGHTR), IA4, ICAM-1, ICOS, Ig alpha (CD79a), IL2R beta, IL2R gamma, IL7R alpha, integrin, ITGA4, ITGA6, ITGAD, ITGAE, ITGAL, ITGAM, ITGAX, ITGB2, ITGB7, ITGB1, KIRDS2, LAT, LFA-1, LIGHT (tumor necrosis factor superfamily member 14; TNFSF14), LTBR, Ly9 (CD229), lymphocyte function-associated antigen-1 (LFA-1 (CD11a/CD18), MHC class I molecule, NKG2C, NKG2D, NKp30, NKp44, NKp46, NKp80 (KLRF1), OX40, PAG/Cbp, PD-1, PSGL1, SELPLG (CD162), signaling lymphocytic activation molecule, SLAM (SLAMF1; CD150; IPO-3), SLAMF4 (CD244; 2B4), SLAMF6 (NTB-A; Ly108), SLAMF7, SLP-76, TNF, TNFr, TNFR2, Toll ligand receptor, TRANCE/RANKL, VLA1, or VLA-6, or fragments, truncations, or combinations thereof.

The terms “reducing” and “decreasing” are used interchangeably herein and indicate any change that is less than the original. “Reducing” and “decreasing” are relative terms, requiring a comparison between pre- and post-measurements. “Reducing” and “decreasing” include complete depletions. Similarly, the term “increasing” indicates any change that is higher than the original value. “Increasing,” “higher,” and “lower” are relative terms, requiring a comparison between pre- and post-measurements and/or between reference standards. In some embodiments, the reference values are obtained from those of a general population, which could be a general population of patients. In some embodiments, the reference values come quartile analysis of a general patient population.

“Treatment” or “treating” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease. In some embodiments, “treatment” or “treating” includes a partial remission. In another embodiment, “treatment” or “treating” includes a complete remission.

As used herein, the term “polyfunctional T cells” refers to cells co-secreting at least two proteins from a pre-specified panel per cell coupled with the amount of each protein produced (i.e., combination of number of proteins secreted and at what intensity). In some embodiments, a single cell functional profile is determined for each evaluable population of engineered T cells. Profiles may be categorized into effector (Granzyme B, IFN-γ, MIP-1α, Perforin, TNF-α, TNF-β), stimulatory (GM-CSF, IL-2, IL-5, IL-7, IL-8, IL-9, IL-12, IL-15, IL-21), regulatory (IL-4, IL-10, IL-13, IL-22, TGF-β1, sCD137, sCD40L), chemo attractive (CCL-11, IP-10, MIP-1β, RANTES), and inflammatory (IL-1b, IL-6, IL-17A, IL-17F, MCP-1, MCP-4) groups. In some embodiments, the functional profile of each cell enables the calculation of other metrics, including a breakdown of each sample according to cell polyfunctionality (i.e., what percentage of cells are secreting multiple cytokines versus non-secreting or monofunctional cells), and a breakdown of the sample by functional groups (i.e., which mono- and polyfunctional groups are being secreted by cells in the sample, and their frequency).

As used herein, the term “quartile” or “quadrant” is a statistical term describing a division of observations into four defined intervals based upon the values of the data and how they compare to the entire set of observations.

As used herein, the term “Study day 0” is defined as the day the subject received the first CART cell infusion. The day prior to study day 0 will be study day −1. Any days after enrollment and prior to study day −1 will be sequential and negative integer-valued.

As used herein, the term “objective response” refers to complete response (CR), partial response (PR), or non-response. Criteria are based on the revised IWG Response Criteria for Malignant Lymphoma.

As used herein, the term “complete response” refers to complete resolution of disease, which becomes not detectable by radio-imaging and clinical laboratory evaluation. No evidence of cancer at a given time.

As used herein, the term “partial response” refers to a reduction of greater than 30% of tumor without complete resolution. Criteria are based on the revised IWG Response Criteria for Malignant Lymphoma where PR is defined as “At least a 50% decrease in sum of the product of the diameters (SPD) of up to six of the largest dominant nodes or nodal masses. These nodes or masses should be selected according to all of the following: they should be clearly measurable in at least 2 perpendicular dimensions; if possible they should be from disparate regions of the body; and they should include mediastinal and retroperitoneal areas of disease whenever these sites are involved

As used herein, the term “non-response” refers to the subjects who had never experienced CR or PR post CAR T cell infusion.

As used herein, the term “durable response” refers to the subjects who were in ongoing response at least by one year follow up post CAR T cell infusion 6 months f/u is utilized only for Z1, C3 as there is no longer f/u available for this cohort. Nevertheless, the conclusions remain same.

As used herein, the term “relapse” refers to the subjects who achieved a complete response (CR) or partial response (PR) and subsequently experienced disease progression.

As used herein, the expansion and persistence of CAR T cells in peripheral blood may be monitored by qPCR analysis, for example using CAR-specific primers for the scFv portion of the CAR (e.g., heavy chain of a CD19 binding domain) and its hinge/CD28 transmembrane domain. Alternatively, it may be measured by enumerating CAR cells/unit of blood volume.

As used herein, the scheduled blood draw for CAR T cells may be before CAR T cell infusion, Day 7, Week 2 (Day 14), Week 4 (Day 28), Month 3 (Day 90), Month 6 (Day 180), Month 12 (Day 360), and Month 24 (Day 720).

As used herein, the “peak of CAR T cell” is defined as the maximum absolute number of CAR+PBMC/μL in serum attained after Day 0.

As used herein, the “time to Peak of CAR T cell” is defined as the number of days from Day 0 to the day when the peak of CAR T cell is attained.

As used herein, the “Area Under Curve (AUC) of level of CAR T cell from Day 0 to Day 28” is defined as the area under the curve in a plot of levels of CAR T cells against scheduled visits from Day 0 to Day 28. This AUC measures the total levels of CAR T cells overtime.

As used herein, the scheduled blood draw for cytokines is before or on the day of conditioning chemotherapy (Day −5), Day 0, Day 1, Day 3, Day 5, Day 7, every other day if any through hospitalization, Week 2 (Day 14), and Week 4 (Day 28).

As used herein, the “baseline” of cytokines is defined as the last value measured prior to conditioning chemotherapy.

As used herein, the fold change from baseline at Day X is defined as

$$\frac{Cytokine level at Day X - Basilne}{Baseline}$$

As used herein, the “peak of cytokine post baseline” is defined as the maximum level of cytokine in serum attained after baseline (Day −5) up to Day 28.

As used herein, the “time to peak of cytokine” post CAR T cell infusion is defined as the number of days from Day 0 to the day when the peak of cytokine was attained.

As used herein, the “Area Under Curve (AUC) of cytokine levels” from Day −5 to Day 28 is defined as the area under the curve in a plot of levels of cytokine against scheduled visits from Day −5 to Day 28. This AUC measures the total levels of cytokine overtime. Given the cytokine and CAR+ T cell are measured at certain discrete time points, the trapezoidal rule may be used to estimate the AUCs.

As used herein, the term “negligible impact” and its metes and bounds would be readily understood by one of ordinary skill in the art. By way of non-limiting example, one of ordinary skill in the art would understand that a negligible impact could mean one or more of: a statistically insignificant effect on the expression of the chimeric antigen receptor, a statistically insignificant effect on the therapeutic efficacy of the chimeric antigen receptor, a statistically insignificant effect on the toxicity of the chimeric antigen receptor on a patient, an impact on expression and/or efficacy and/or toxicity of the chimeric antigen receptor that is not beyond a predetermined threshold value of expression and/or efficacy and/or toxicity for the chimeric antigen receptor.

It will be appreciated that chimeric antigen receptors (CARs or CAR-Ts) are, and T cell receptors (TCRs) may, be genetically engineered receptors. These engineered receptors may be readily inserted into and expressed by immune cells, including T cells in accordance with techniques known in the art. With a CAR, a single receptor may be programmed to both recognize a specific antigen and, when bound to that antigen, activate the immune cell to attack and destroy the cell bearing that antigen. When these antigens exist on tumor cells, an immune cell that expresses the CAR may target and kill the tumor cell.

CARs may be engineered to bind to an antigen (such as a cell-surface antigen) by incorporating an antigen binding molecule that interacts with that targeted antigen. An “antigen binding molecule” as used herein means any protein that binds a specified target molecule. Antigen binding molecules include, but are not limited to antibodies and binding parts thereof, such as immunologically functional fragments. Peptibodies (i.e., Fc fusion molecules comprising peptide binding domains) are another example of suitable antigen binding molecules.

Preferably, target molecules may include, but are not limited to, blood borne cancer-associated antigens. Non-limiting examples of blood borne cancer-associated antigens include antigens associated with one or more cancers selected from the group consisting of acute myeloid leukemia (AML), chronic myelogenous leukemia (CML), chronic myelomonocytic leukemia (CMML), juvenile myelomonocytic leukemia, atypical chronic myeloid leukemia, acute promyelocytic leukemia (APL), acute monoblastic leukemia, acute erythroid leukemia, acute megakaryoblastic leukemia, lymphoblastic leukemia, B-lineage acute lymphoblastic leukemia, B-cell chronic lymphocytic leukemia, B-cell non-Hodgkin's lymphoma, myelodysplastic syndrome (MDS), myeloproliferative disorder, myeloid neoplasm, myeloid sarcoma), and Blastic Plasmacytoid Dendritic Cell Neoplasm (BPDCN).

In some embodiments, the antigen is selected from a tumor-associated surface antigen, such as 5T4, alphafetoprotein (AFP), B7-1 (CD80), B7-2 (CD86), BCMA, B-human chorionic gonadotropin, CA-125, carcinoembryonic antigen (CEA), carcinoembryonic antigen (CEA), CD123, CD133, CD138, CD19, CD20, CD22, CD23, CD24, CD25, CD30, CD33, CD34, CD4, CD40, CD44, CD56, CD8, CLL-1, c-Met, CMV-specific antigen, CSPG4, CTLA-4, disialoganglioside GD2, ductal-epithelial mucine, EBV-specific antigen, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (HMW-MAA), HIV-1 envelope glycoprotein gp41, HPV-specific antigen, human telomerase reverse transcriptase, IGFI receptor, IGF-II, IL-11Ralpha, IL-13R-a2, Influenza Virus-specific antigen; CD38, insulin growth factor (IGF1)-1, intestinal carboxyl esterase, kappa chain, LAGA-1a, lambda chain, Lassa Virus-specific antigen, lectin-reactive AFP, lineage-specific or tissue specific antigen such as CD3, MAGE, MAGE-A1, major histocompatibility complex (MHC) molecule, major histocompatibility complex (MHC) molecule presenting a tumor-specific peptide epitope, M-CSF, melanoma-associated antigen, mesothelin, mesothelin, MN-CA IX, MUC-1, mut hsp72, mutated p53, mutated p53, mutated ras, neutrophil elastase, NKG2D, Nkp30, NY-ESO-1, p53, PAP, prostase, prostase specific antigen (PSA), prostate carcinoma tumor antigen-1 (PCTA-1), prostate-specific antigen, prostein, PSMA, RAGE-1, ROR1, RU1, RU2 (AS), surface adhesion molecule, surviving and telomerase, TAG-72, the extra domain A (EDA) and extra domain B (EDB) of fibronectin and the A1 domain of tenascin-C (TnC A1), thyroglobulin, tumor stromal antigens, vascular endothelial growth factor receptor-2 (VEGFR2), virus-specific surface antigen such as an HIV-specific antigen (such as HIV gp120), as well as any derivate or variant of these surface markers.

In some embodiments, target molecules may include viral infection-associated antigens. Viral infections of the present disclosure may be caused by any virus, including, for example, HIV. This list of possible target molecules is not intended to be exclusive.

The TCRs of the present disclosure may bind to, for example, a tumor-associated antigen. As used herein, “tumor-associated antigen” refers to any antigen that is associated with one or more cancers selected from the group consisting of: adrenocortical carcinoma, anal cancer, bladder cancer, bone cancer, brain cancer, breast cancer, carcinoid cancer, carcinoma, cervical cancer, colon cancer, endometrial cancer, esophageal cancer, extrahepatic bile duct cancer, extracranial germ cell cancer, eye cancer, gallbladder cancer, gastric cancer, germ cell tumor, gestational trophoblastic tumor, head and neck cancer, hypopharyngeal cancer, islet cell carcinoma, kidney cancer, large intestine cancer, laryngeal cancer, leukemia, lip and oral cavity cancer, liver cancer, lung cancer, lymphoma, malignant mesothelioma, Merkel cell carcinoma, mycosis fungoides, myelodysplastic syndrome, myeloproliferative disorders, nasopharyngeal cancer, neuroblastoma, oral cancer, oropharyngeal cancer, osteosarcoma, ovarian epithelial cancer, ovarian germ cell cancer, pancreatic cancer, paranasal sinus and nasal cavity cancer, parathyroid cancer, penile cancer, pituitary cancer, plasma cell neoplasm, prostate cancer, rhabdomyosarcoma, rectal cancer, renal cell cancer, transitional cell cancer of the renal pelvis and ureter, salivary gland cancer, Sezary syndrome, skin cancers, small intestine cancer, soft tissue sarcoma, stomach cancer, testicular cancer, thymoma, thyroid cancer, urethral cancer, uterine cancer, vaginal cancer, vulvar cancer, and Wilms' tumor.

In certain embodiments, the present disclosure may be suitable for target molecule to hematologic cancer. In some embodiments, the cancer is of the white blood cells. In other embodiments, the cancer is of the plasma cells. In some embodiments, the cancer is leukemia, lymphoma, or myeloma. In certain embodiments, the cancer is acute lymphoblastic leukemia (ALL) (including non T cell ALL), acute lymphoid leukemia (ALL), and hemophagocytic lymphohistocytosis (HLH)), B cell prolymphocytic leukemia, B-cell acute lymphoid leukemia (“BALL”), blastic plasmacytoid dendritic cell neoplasm, Burkitt's lymphoma, chronic lymphocytic leukemia (CLL), chronic myelogenous leukemia (CML), chronic myeloid leukemia (CML), chronic or acute granulomatous disease, chronic or acute leukemia, diffuse large B cell lymphoma, diffuse large B cell lymphoma (DLBCL), follicular lymphoma, follicular lymphoma (FL), hairy cell leukemia, hemophagocytic syndrome (Macrophage Activating Syndrome (MAS), Hodgkin's Disease, large cell granuloma, leukocyte adhesion deficiency, malignant lymphoproliferative conditions, MALT lymphoma, mantle cell lymphoma, Marginal zone lymphoma, monoclonal gammapathy of undetermined significance (MGUS), multiple myeloma, myelodysplasia and myelodysplastic syndrome (MDS), myeloid diseases including but not limited to acute myeloid leukemia (AML), non-Hodgkin's lymphoma (NHL), plasma cell proliferative disorders (e.g., asymptomatic myeloma (smoldering multiple myeloma or indolent myeloma), plasmablastic lymphoma, plasmacytoid dendritic cell neoplasm, plasmacytomas (e.g., plasma cell dyscrasia; solitary myeloma; solitary plasmacytoma; extramedullary plasmacytoma; and multiple plasmacytoma), POEMS syndrome (Crow-Fukase syndrome; Takatsuki disease; PEP syndrome), primary mediastinal large B cell lymphoma (PMBCL), small cell- or a large cell-follicular lymphoma, splenic marginal zone lymphoma (SMZL), systemic amyloid light chain amyloidosis, T-cell acute lymphoid leukemia (TALL), T-cell lymphoma, transformed follicular lymphoma, Waldenstrom macroglobulinemia, or a combination thereof.

The TCRs of the present disclosure may also bind to a viral infection-associated antigen. Viral infection-associated antigens include antigens associated with any viral infection, including, for example, viral infection caused by HIV.

Various embodiments are described in further detail in the following subsections.

### Variant Creation

Cell therapy products, including autologous, allogeneic, neoantigen and other types of products, have the potential to express RNA and protein sequences other than the desired or transfected gene(s). Expression of these non-standard sequences (referred to as variants) can occur due to at least two different mechanisms.

The first mechanism, RNA splicing, can lead to the product expressing variants even when it has been transfected with the intended gene sequence, as the product is transcribed into an RNA product that is then spliced. RNA splicing can also cause the product to be transfected (at the DNA level) with a variant sequence if the product manufacturing process involves reverse transcription, since RNA may be spliced prior to being reverse transcribed into DNA that is then transfected into the cell.

The second mechanism, homologous recombination, occurs when reverse transcription is part of the manufacturing process. In this phenomenon, an actively transcribing reverse transcriptase “jumps” between two highly similar (or identical) sequences of the RNA template, thus skipping the intervening sequence and creating a non-standard DNA transcript that may later be transfected into the product. Because this mechanism is dependent on highly similar sequences in template, it is a particular risk for bicistronic CAR products where the two CARs may have some domains (e.g. co-stimulatory domains) that are identical.

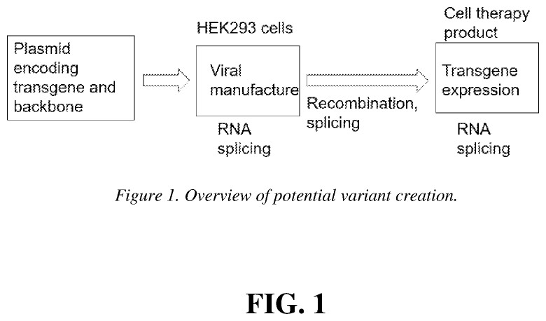

Considering both of these mechanisms and looking at a conventional CAR-T cell manufacturing procedure using lentiviral vectors, we find several points where variants may be created. First, and as shown in the example of FIG. 1, HEK293 cells may be transfected with product and lentiviral encoding plasmids to produce lentiviral vectors. RNA produced by the HEK293 cells may be spliced prior to packaging into these vectors. Second, lentiviral vectors are used to transfect T cells. This involves reverse transcription of the product sequence, prior to its integration into the T cell genome, during which homologous recombination may occur. Third, the transfected T cell (now a CAR-T cell) expresses the CAR(s) and may splice the expressed CAR mRNA prior to its translation into protein.

Regardless of the origin, variants are undesirable. Variants in cell therapy products may have little to no efficacy. Also, variants may cause cell therapy products to recognize a different antigen and thus cause off target toxicity. Below is a description of different embodiments for a general pipeline or process for prediction, detection and elimination of variants to prevent these outcomes from occurring.

An embodiment of the disclosure relates to a method for detecting and replacing a sequence which may cause an undesired variant in a gene construct. Such a method includes: performing an in-silico analysis of the gene construct to detect a presence of the sequence which may cause the undesired variant; replacing the detected sequence which may cause the undesired variant with an alternative sequence, where the alternative sequence is derived comprising synonymous codon substitution; measuring a frequency percentage of the undesired variant expressed by the gene construct comprising performing an in-vivo analysis of one or more genes expressed by the gene construct comprising performing a RNA-sequencing analysis of an RNA product transcribed from the gene construct, where the frequency percentage of the undesired variant is determined at least in part by using a splice-aware aligner from the RNA-sequencing analysis; and repeating the in-silico analysis and replacing steps if the frequency percentage of the undesired variant in the gene product from the in-vivo analysis is greater than a predetermined value of acceptable frequency percentage of the undesired variant.

An embodiment of the disclosure relates to the method above, where the gap-aware alignment includes using at least two separate aligners.

An embodiment of the disclosure relates to any of the methods above, where the in-silico analysis further includes: detecting at least one of a plurality of homologous sequences and a plurality of identical sequences within the gene construct, where the at least one of the plurality of homologous sequences and the plurality of identical sequences may cause an undesired variant in the gene construct; and replacing any such detected plurality of homologous sequences and plurality of identical sequences by performing a step of synonymous codon substitution.

An embodiment of the disclosure relates to any of the methods above, where the in-silico analysis further includes calculating a matrix of subsection combinations from the gene construct and acquiring a Hamming distance for each of the subsection combinations.

An embodiment of the disclosure relates to any of the methods above, where the in-silico analysis further includes substituting a plurality of random synonymous codons in the gene construct with a plurality of alternative sequences such that plurality of alternative sequences increases a sum over the matrix.

An embodiment of the disclosure relates to any of the methods above, where the gene construct includes a sequence encoding a chimeric antigen receptor.

An embodiment of the disclosure relates to any of the methods above, where the predetermined value of acceptable frequency percentage of undesired variant is determined based on whether the undesired variant is associated with at least one of whether the undesired variant negatively impacts exportation of the chimeric antigen receptor to a cell surface, whether the undesired variant is associated with changes to a binding domain of the chimeric antigen receptor, and whether the undesired variant has been previously characterized as causing a negligible impact on the expression or function of the chimeric antigen receptor.

An embodiment of the disclosure relates to any of the methods above, where the predetermined value of acceptable frequency percentage of the undesired variant is 0.1% if the undesired variant negatively impacts exportation of the chimeric antigen receptor to a cell surface, and where the predetermined value of acceptable frequency percentage of the undesired variant is 0.01% if the undesired variant is associated with changes to a binding domain of the chimeric antigen receptor.

An embodiment of the disclosure relates to any of the methods above, where the repeating the in-silico analysis and replacing steps is not performed if the undesired variant has been previously characterized as causing a negligible impact on the expression or function of the chimeric antigen receptor.

An embodiment of the disclosure relates to any of the methods above, further including steps of identifying and removing a subpopulation of high-frequency variants and identifying a subpopulation of low-frequency variants, and where the in vivo analysis further includes conducting an analysis to determine whether the subpopulation of low-frequency variants should be replaced.

An embodiment of the disclosure relates to any of the methods above, where if an undesired variant is detected and does not meet any of the abovementioned conditions or requirements, then in-silico analysis and sequence removal steps are repeated to attempt to eliminate the undesired variant, and/or the undesired variant is further characterized in additional studies to assess a risk to potential patients.

An embodiment of the disclosure relates to a method for creating a gene product used in cell therapy. Such a method includes the steps of: performing an in-silico analysis on a gene construct encoding the gene product to identify and alter a sequence that may causes an undesired variant; replacing the detected sequence which may cause the undesired variant with an alternative sequence, where the alternative sequence is derived comprising synonymous codon substitution; measuring a frequency percentage of the undesired variant expressed by the gene construct comprising performing an in-vivo analysis of one or more genes expressed by the gene construct comprising performing a RNA-sequencing analysis of an RNA product transcribed from the gene construct, where the frequency percentage of the undesired variant is determined at least in part by using a splice-aware aligner from the RNA-sequencing analysis; repeating the in-silico and replacing steps to create a new gene construct if the frequency percentage of the undesired variant in the gene product from the in-vivo analysis is greater than a predetermined value of acceptable frequency percentage of the undesired variant; and measuring a frequency percentage of the undesired variant expressed by the new gene construct comprising performing an in-vivo analysis of one or more genes expressed by the new gene construct comprising performing a RNA-sequencing analysis of an RNA product transcribed from the new gene construct, where the frequency percentage of the undesired variant is determined at least in part by using a splice-aware aligner from the RNA-sequencing analysis.

An embodiment of the disclosure relates to the method above, where the gap-aware alignment includes using at least two separate aligners.

An embodiment of the disclosure relates to any of the methods above, where the in-silico analysis further includes the steps of: detecting at least one of a plurality of homologous sequences and a plurality of identical sequences within the gene construct, where the at least one of the plurality of homologous sequences and the plurality of identical sequences may cause an undesired variant in the gene construct; and replacing any such detected plurality of homologous sequences and plurality of identical sequences comprising a step of synonymous codon substitution.

An embodiment of the disclosure relates to any of the methods above, where the in-silico analysis further includes calculating a matrix of subsection combinations from the gene construct and acquiring a Hamming distance for each of the subsection combinations.

An embodiment of the disclosure relates to any of the methods above, where the in-silico analysis further includes substituting a plurality of random synonymous codons in the gene construct with a plurality of alternative sequences such that plurality of alternative sequences increases a sum over the matrix.

An embodiment of the disclosure relates to any of the methods above, where the gene construct includes a sequence encoding a chimeric antigen receptor.

An embodiment of the disclosure relates to any of the methods above, where the predetermined value of acceptable frequency percentage of undesired variant is determined based on whether the undesired variant is associated with at least one of whether the undesired variant negatively impacts exportation of the chimeric antigen receptor to a cell surface, whether the undesired variant is associated with changes to a binding domain of the chimeric antigen receptor, and whether the undesired variant has been previously characterized as causing a negligible impact on the expression or function of the chimeric antigen receptor.

An embodiment of the disclosure relates to any of the methods above, where the predetermined value of acceptable frequency percentage of the undesired variant is 0.1% if the undesired variant negatively impacts exportation of the chimeric antigen receptor to a cell surface, and where the predetermined value of acceptable frequency percentage of the undesired variant is 0.01% if the undesired variant is associated with changes to a binding domain of the chimeric antigen receptor.

An embodiment of the disclosure relates to any of the methods above, where the repeating the in-silico analysis and replacing steps is not performed if the undesired variant has been previously characterized as causing a negligible impact on the expression or function of the chimeric antigen receptor.

An embodiment of the disclosure relates to any of the methods above, further including the steps of identifying and removing a subpopulation of high-frequency variants and identifying a subpopulation of low-frequency variants, and where the in vivo analysis further includes conducting an analysis to determine whether the subpopulation of low-frequency variants should be replaced.

An embodiment of the disclosure relates to any of the methods above, where if an undesired variant is detected and does not meet any of the abovementioned conditions or requirements, then in-silico analysis and sequence removal steps are repeated to attempt to eliminate the undesired variant, and/or the undesired variant is further characterized in additional studies to assess a risk to potential patients.

An embodiment of the disclosure relates to a method for reducing a risk that a gene product used in cell therapy carries a risk of reduced efficacy or toxicity due to production of an undesired variant. Such a method includes the steps of: performing an in-silico analysis of a gene construct encoding the gene product to detect a presence of a sequence which may cause the undesired variant; replacing the detected sequence which may cause the undesired variant with an alternative sequence, where the alternative sequence is derived comprising synonymous codon substitution; measuring a frequency percentage of the undesired variant expressed by the gene construct comprising performing an in-vivo analysis of one or more genes expressed by the gene construct comprising performing a RNA-sequencing analysis of an RNA product transcribed from the gene construct, where the frequency percentage of the undesired variant is determined at least in part by using a splice-aware aligner from the RNA-sequencing analysis; and repeating the in-silico analysis and replacing steps if the frequency percentage of the undesired variant in the gene product from the in-vivo analysis is greater than a predetermined value of acceptable frequency percentage of the undesired variant.

### Variant Prediction, Detection, and Elimination

One embodiment discloses a general pipeline for dealing with variants. This pipeline depends both on algorithms to identify and remove potential variant causing sequences, and sequencing and alignment methods to test for the presence of variants. The algorithmic and sequencing based portions of this pipeline are described in further detail in subsequent sections.

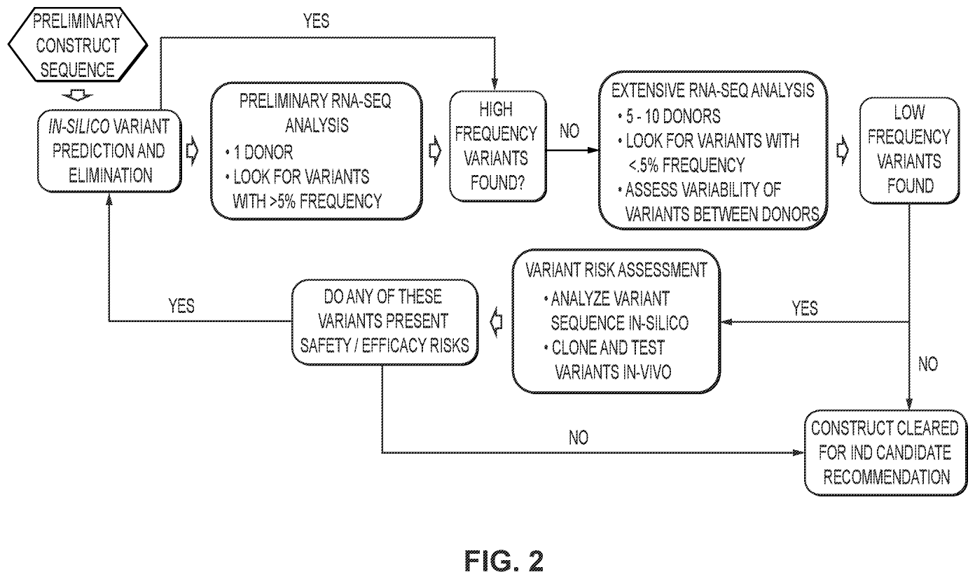

An outline of one embodiment of a pipeline is provided in FIG. 2. This pipeline uses a looping logic where efforts are made to identify and remove variant causing sequences in-silico, followed by physical testing and more in-silico work if variants are found. Variant detection is done in multiple sequencing steps so extensive effort is not put into construct sequences that can be easily identified as producing variants.

The pipeline is structured to have flexibility in this embodiment. The frequency cutoffs shown in FIG. 2 are examples and alternative methods of determining if a variant needs to be addressed (discussed in the sequencing section) can be used. Likewise, it is possible to increase/decrease the number of donors in the sequence step or add/remove additional sequence steps (for example, sequencing of lentiviral vectors) to maximize efficiency or look even more deeply for variants.

With reference to FIG. 2, prior to any physical work, the planned construct sequence is subject to algorithms designed to identify splice sites and highly homologous sequences (which could cause homologous recombination) in one embodiment. If either of these are identified, other algorithms are used to remove them via synonymous codon substitution (the construct protein sequence does not change). Once this initial in-silico screening and modification is completed, a small test batch of product using a single donor is created and RNA-sequence of this batch is used to identify any high frequency (e.g., greater than about 5%) variants. If high frequency variants are found, the in-silico screening and modification process is repeated, guided by knowledge of the identified variants, before the small batch creation and RNA-sequence is reattempted. If no high frequency variants are found, a larger batch of test product, using 5-10 donors, is created and RNA-sequence of this batch is used to identify any low frequency (<5%) variants. If any of these variants are found, the sequence and expression level can be analyzed in-silico to determine if the variant present a safety/efficacy risk. As an example of this assessment, a variant that occurs at only 1% of the product and results in CAR with a spliced co-stimulatory domain is unlikely to be an issue, as this will probably just result in a very small decrease in efficacy. In contrast, splicing in the ScFv domain of the CAR, even at low a percentage, may result in off target binding and thus toxicity. If a safety/efficacy risk cannot be ruled out, products expressing these particular variants can be created and physically tested. If these tests suggest an issue with safety/efficacy or cannot be performed, the construct sequence can be redesigned in-silico and the entire process restarted. Otherwise, if low frequency variants are not found, or confirmed to present no risk to safety/efficacy, the construct sequence can be cleared for further development.

### Splice Site Prediction and Removal Algorithms

During the in-silico step of this pipeline, the SpliceAI—Jaganathan et al. Cell 2019 (https://github.com/Illumina/SpliceAI) and MaxEntScan—Yeo and Burge, J Comput Biol 2003 (http://hollywood.mit.edu/burgelab/maxent/Xmaxentscan scoreseq.html) algorithms may be used to identify potential donor and acceptor splice sites in one embodiment. Other algorithms may be used in other embodiments.

In one embodiment, if sites in the construct are identified by either of these algorithms, the sites can be modified, using synonymous codon substitution, until the variant is no longer detected, or its predicted splicing likelihood is greatly reduced. This step leaves the construct protein sequence unchanged. Likewise, if sequencing has already been performed and variants detected, these algorithms can be used to determine if a variant might be due to splicing and use synonymous codon substitution to reduce the likelihood of the variant occurring.

### Homology Identification and Removal Algorithms

Since homologous recombination is driven by highly similar sequences in the construct, the present disclosure describes algorithms and tools to help spot and remove these sequences. Just as with the splice site removal algorithms, these algorithms are run in the initial in-silico step and can be rerun after sequencing steps if sequencing indicates homologous recombination.

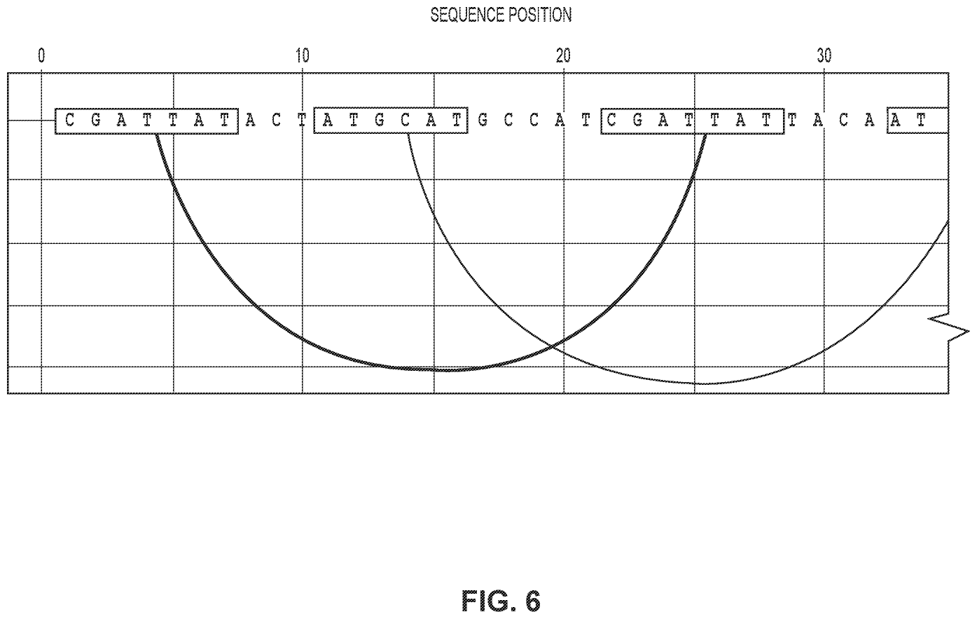

In order to identify sequences that are likely to cause homologous recombination, one embodiment includes a Repeat Finder tool. This tool analyzes and displays a construct sequence and draws arches connecting all pairs of identical sequences longer than a given (user set) length. Alternatively, this tool can connect sequence pairs of a given size and with a given level of similarity as judged by a user set Levenshtein distance. An example of a screen shot showing an output display from the Repeat Finder tool is shown in FIG. 6. The thickness of an arch is dependent on the length of the identical sequences it links. By looking for groups of arches clustered together or thick arches, users can easily identify highly similar sequences, even when they are not identical.

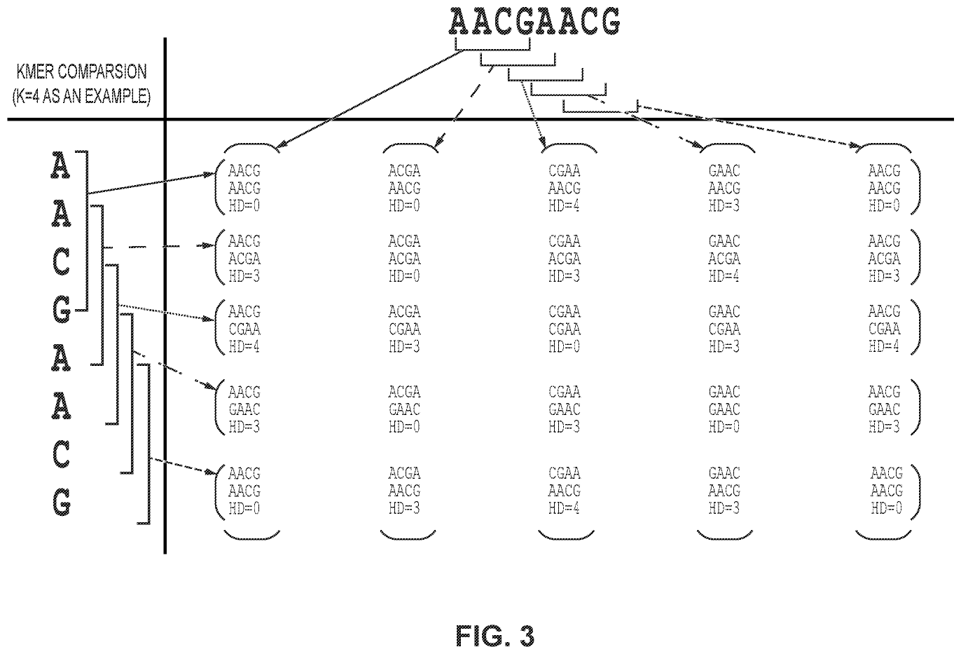

In order to reduce the similarity between all subsections of a given construct, one embodiment includes a Sequence Diverger tool. This tool takes a construct sequence of length n (in base pairs) and user selected subsection size of k (also in base pairs). The tool then creates a matrix that is n−k+1 in both dimensions. Each position (x,y) in this matrix corresponds to a pair of subsections starting at bases x and y of the construct sequence. The value of this position is the Hamming distance between these two subsections. An example of the matrix is shown in FIG. 3. With this design, the sum of the matrix effectively describes the construct sequence's similarity to itself. A construct sequence with many highly similar subsections will have a smaller sum than one where most subsections are different from each other. This means that decreasing the similarity between all subsections of the construct, and thus the likelihood of homologous recombination events, is a matter of increasing the sum of this matrix. The Sequence Diverger achieves this by making random synonymous codon substitutions throughout the construct sequence and only keeping those that increase the sum of the matrix. A user specified number of substitutions are made before the algorithm is terminated and the modified sequence is returned. The user can track the sum of the matrix with respect to the number of substitutions attempted and thus get a sense of how many steps are needed.

As shown in the example of FIG. 3, for a given sequence (AACGAACG) and given subsection size (4) the Hamming Distance (HD) is calculated for all possible subsection pairs. The sum of the matrix describes how similar different subsections of the sequence are to each other and thus maximizing this sum should decrease the potential for homologous recombination.

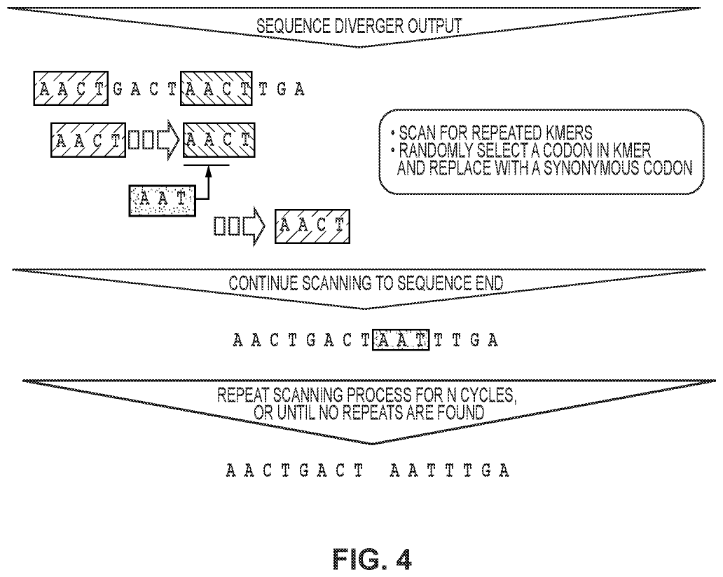

In another embodiment, a Repeat Remover tool may be used to help prevent the construct sequence from containing any identical subsections which could cause homologous recombination. This tool takes a construct sequence and user selected subsection size k (in base pairs). In one embodiment, the Repeat Remover tool then undergoes a process where each subsection of size k, starting from construct position 1, is compared to all other subsections in the construct. If an identical subsection is found, a random synonymous codon substitution in that subsection is used to eliminate the similarity. This process is repeated in cycles until the entire construct is scanned without any identical subsections being found or a user defined number of cycles is reached. An example of this is shown in FIG. 4. Multiple cycle repeats may be necessary because it is possible for a synonymous codon substitution that is made to eliminate one pair of identical sequences to introduce another.

As shown in FIG. 4, the Repeat Remover tool compares all subsections of a user selected size (e.g., 6 to 10 base pairs) to each other to see if they are identical. If an identical pair is found, a synonymous codon substitution is used to remove it. This continues until all identical pairs have been removed or a user defined number of cycles is reached in this embodiment.

Although they have the same goal of removing the highly similar sequences that can cause homologous recombination, the Sequence Diverger and Repeat Remover tools have different approaches and potentially different outcomes. Repeat Remover is focused on eliminating identical sequences and may do so at the expense of creating highly similar (but non-identical) sequences. In contrast, Sequence Diverger is focused on globally reducing sequence similarity and may do this at the expense of leaving a few identical sequences in place. The in-silico step of one embodiment could be done with both tools in either order or with only one, depending on what works best for the particular construct being developed. In other embodiments, neither tool may be used in the process and instead manual removal of highly similar sequences identified via Repeat Remover (or Repeat Visualizer) may be used.

These embodiments are flexible in that the tools described above can be used individually or in combination with one another, and additional tools or features may be added. For example, all three tools can be set to ignore highly similar/identical sequences that are within a given distance of each other. This is relevant because there are some indications that homologous recombination events require a minimum distance between the highly similar sequences to occur. It is also possible to set Sequence Diverger and Repeat Remover tools so that certain regions of the construct are not changed in case these regions are known to be highly sensitive to codon usage. Finally, it would be possible to modify the Sequence Diverger tool to accept or reject synonymous codon substitutions based on a simulated annealing logic, rather than only accepting those that increase the matrix sum. This may help the tool better find the global maxima.

In certain embodiments, the Repeat Finder, Sequence Diverger, and Repeat Remover tools may be individual computer modules. In other embodiments, these three tools may be programmed into a single module or computer.

### Sequencing Based Variant Detection

There is some flexibility as to how RNA sequencing to identify variants can be performed. It is desirable that the sequencing be of sufficient quality and depth to allow variants to be seen. As an example, CAR-T products with a HiSeq 2500 sequencing lane, a depth of ˜300 million reads per sample and paired end reads of 150 bp length may be sequenced in one embodiment.

Following sequencing, reads should be aligned to the construct using one or more splice aware aligners. There is flexibility in this step based on precise alignment parameters and what aligners are used. This embodiment has found good results using three different aligners (STAR, HISAT2, and TopHat2) simultaneously, as this helps identify and ignore the mistakes of any one aligner (discussed later).

Following alignment in one embodiment, all reads with gapped alignments (as these are the ones that would indicate variants) are extracted and product protein sequences that they would lead to are translated. The percentage of each variant (protein sequences differing from the intended sequence) in the sample is calculated using the formula:

$$x = \frac{100 * R * n}{{\sum }_{i = 1}^{n} d}$$

Where x is the percentage, R is the number of reads supporting the variant, n is the number of bases in the open reading frame (ORF) and d is the read depth (all reads) at each position in the ORF.

Following the assignment of reads and percentage of sample to each variant, the final step of this embodiment is to determine what variants require further investigation. This step allows for flexibility and consideration should be given to if this is an initial small screen to identify highly expressed variants or a large one to identify all variants (see FIG. 2).

One option is to establish a cutoff based on the number of reads. For example, a sample could be considered positive for a variant if it has 5 or more reads supporting that variant. Another option would be to consider if the percentage of the sample supporting the variant (as shown in FIG. 2) is above a certain cutoff. In both cases, if samples from multiple donors are used, a secondary cutoff based on multiple donors can be established. For example, only variants that are positive in samples from a given number or percentage of donors will be considered. In another embodiment, it is possible to use a statistical test, such as the Wilcox Rank Sum Test, where each donor sample is considered an independent experiment, to determine if the rate of donors positive for a given variant significantly differs from 0. Finally, if multiple aligners are used, a cutoff based on them can be used to address inaccuracies in any one pipeline. For example, a sample might only be considered positive for a variant if it has 5 or more reads supporting that variant in at least two of three aligners. Alternatively, a variant could only be considered if it returns a positive Wilcox Rank Sum Test over multiple donors with at least two pipelines.

After statistical analysis of variants, visual inspection of variant supporting and normal reads in a program such as Integrated Genomics Viewer (IGV) may be conducted. Some apparent variants may be the result of alignment errors that can be spotted with visual inspections. In certain embodiments of the process, in addition to performing the tests just described, the process may include isolating all reads in a sample supporting a particular variant and place them in a single BAM file that can be easily inspected.

### RNA-Seq Profiling and Bioinformatics Analysis

In another embodiment, RNA-sequence profiling and bioinformatics analysis includes a quality control check using FastQC (version 0.11.7) or the like. Using default parameters for FastQC, the gene construct should pass quality control. The profiling and analysis may also include an alignment step. In order to maximize splicing event detection, reads are aligned to the construct sequence using 3 different splice aware aligners: STAR (version 2.7.3a) (PMID: 23104886), HISAT2 (version 2.1.0) (PMID: 31375807), and TopHat2 (version 2.1.0) (PMID: 23618408). Custom alignment indexes corresponding to the construct sequence may be generated for each tool. In one embodiment, the alignment may be performed on the Seven Bridges platform but could be performed on other computing platforms.

In one embodiment, the profiling and analysis may include STAR alignment. The reference index for STAR alignment may be made using the genomeGenerate command with the genomeSAindexNbases parameter set to 5 and all other parameters set to their default values. In one embodiment, alignment in STAR may be done using all default parameters. In one embodiment, the following commands may be used to create the Index and perform the alignment with the STAR tool:

STAR Index Command:

STAR --runMode genomeGenerate --genomeDir
./genomeDir --runThreadN 32 --
genomeS AindexNbases 5
-genomeFastaFiles_construct_name.fa --
limitGenomeGenerateRAM 60000000000

STAR Alignment Command:

STAR --runThreadN 32 --readFilesCommand zcat
--genomeDir ./genomeDir --
limitBAMsortRAM 0 --outSAMtype BAM
Unsorted --readFilesIn R1.fastq.gz
R2.fastq.gz

It should be understood that other commands may be used to create an Index and perform alignment.

Furthermore, the profiling and analysis may include HISAT2 alignment. In one embodiment, the reference index for HISAT2 alignment is made using the hisat2-build command with HISAT2 version 2.0.1. HISAT2 alignment may be run with—no-softclip—no-unal options enabled and the—pen-cansplice and—pen-noncansplice parameters set to 0. All other parameters may be set to their defaults and reads are subsequently sorted using Sambamba (version 0.6.6) (PMID: 25697820). In one embodiment, the following commands may be used to create an index and perform the alignment with the HISAT2 tool:

HISAT2 index command:

hisat2-build-p1 construct_name.fa index/construct_name_HISAT2-2.0.1

HISAT2 alignment command:

hisat2 --met-file metrics.txt --no-softclip --no-unal
-p 20 --pen-cansplice 0 --pen-
noncansplice 0 -x ./index_files_path
-1 R1_001.fastq.gz -2 R2_001.fastq.gz -S
/dev/stdout

It should be understood that other commands may be used to create an Index and perform alignment with the HISAT2 tool.

In one embodiment, the profiling and analysis step may include TopHat2 alignment. The reference index for TopHat2 alignment may be made using the BowTie2-build command (version 2.2.6) (PMID: 21154709), or the like. TopHat2 alignment may be done with all default parameters in one embodiment. The following commands may be used to create an index and perform the alignment with the TopHat2 tool:

BowTie2-build command:

bowtie2-build-f construct.fa./construct_name

TopHat2 alignment:

tophat2—num-threads 1—output-dir./tophat_out—no-coverage-search./construct_name

R1_001.fastq.gz R2_001.fastq.gz

In one embodiment, the analysis step includes processing reads. In this embodiment, aligned reads from each alignment method may be further processed on the Seven Bridges platform. First, the SAMtools (version 1.6) (PMID: 19505943), view function, or the like may be used to remove all non-gapped reads. SAMtools (version 1.9) may be subsequently used to convert the remaining gapped reads in BAM file format into SAM format. Next, an R (version 3.6.2) (https://www.R-project.org/) script (translate_and_group.R), or the like, may be used to translate the nucleotide sequence from each gapped read into its corresponding amino acid sequence. This script also may be used to calculate the number of reads supporting each unique gapped event. Gapped reads with an overhang of less than 10 base pairs may be removed in one embodiment. In one embodiment, this script utilized the Seqinr (version 3.6.1) (ISBN: 978-3-540-35305-8, https://cran.r-project.org/web/packages/seqinr/index.html) library package to translate the gapped DNA sequences into amino acid sequences.
SAMtools command to remove non-gapped reads:
samtools view -h/path/to/input_bam.ext|awk ‘{if($0˜/\hat{ }@/∥$6˜/N/) {print $0}}’| samtools view -Sb→input_bam_gapped.bam
SAMtools command to convert BAM to SAM file format:
samtools view—output-fmt SAM-o hits_gapped.sam hits_gapped.bam

In one embodiment, the last step of the bioinformatic analysis may be performed on Amazon Web Services (AWS) Virtual Private Cloud (VPC) using an EC2 instance running an R (version 3.6.2) script (app.R). This script imported the output file resulting from the s script (translate_and_group.R), along with the BAM output file from the alignment to calculate the prevalence of each gapped event. The formula used for this calculation is:

$$x = \frac{100 * R * n}{{\sum }_{i = 1}^{n} d}$$

Where x is the percentage coverage, R is the number of reads supporting the gap, n is the number of bases in the open reading frame (ORF), and d is the read depth at each position in the ORF).

In one embodiment, the app.R script relies on SAMtools (version 1.10) depth function to calculate coverage for the construct and produce a BAM file (with calls to the SAMtools view function) with the reads from every unique gapped event for visualization purposes. Following this analysis, a gap event may be considered a variant if the event passed the following filtering criteria: The p-value (nonparametric one sample Wilcoxon rank sum test) is <0.01 in 2 out of the 3 methods, this cross-validation was used to minimize method specific artifacts.

The null and alternative hypotheses are:

H₀: μ=0

Hₐ: μ≠0

In one embodiment, a conservative threshold for p-value <0.01 may be selected to minimize false positives rate due to large number of spurious putative variants supported by <5 reads in a single donor and likely the result of sequencing artifacts.

### Report Generation

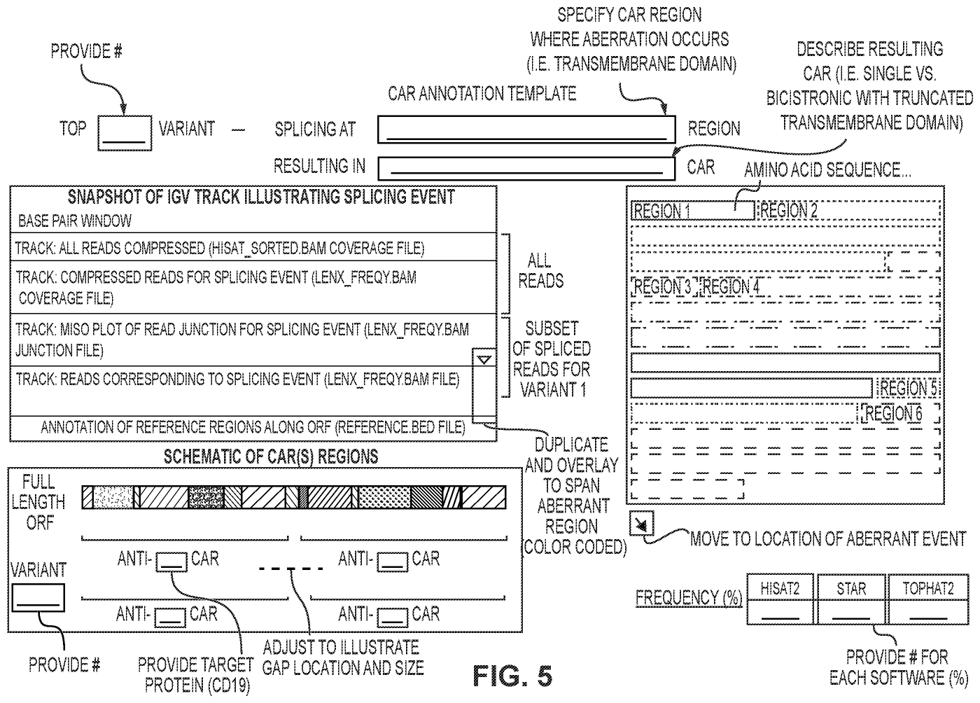

In one embodiment, the variant detection method may generate and display a report for each variant that includes the following information that is shown if FIG. 5: 1) sequence of the expected protein product, 2) variant ID or number, 3) diagram or schematic annotating the changes to the features of the protein product (i.e. CAR), 4) frequency of alignment for each aligner used (e.g. Tophat, HISAT, STAR), 5) P-value of statistical significance of detection (if available), and 6) visualization of reads aligning to the construct.

One skilled in the art will realize the subject matter may be embodied in other specific forms without departing from the spirit or essential characteristics thereof. The foregoing embodiments are therefore to be considered in all respects illustrative rather than limiting of the subject matter described herein.

### EXAMPLES

The following examples disclose an exemplary table of potential software packages that may be used in the RNA-sequencing profiling and bioinformatics analysis, along with exemplary software code portions that may be used in the disclosed process.

### Example 1

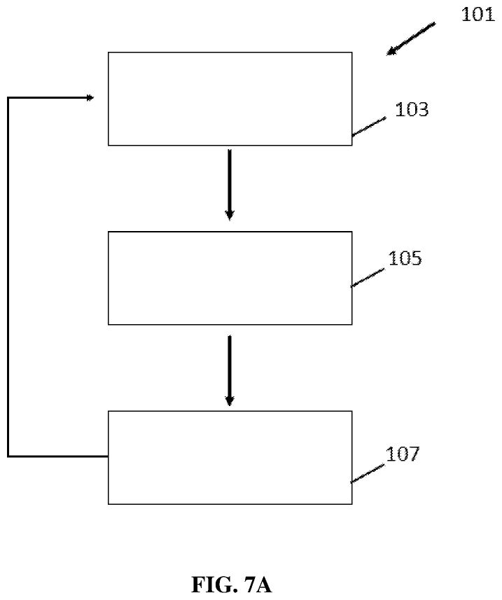

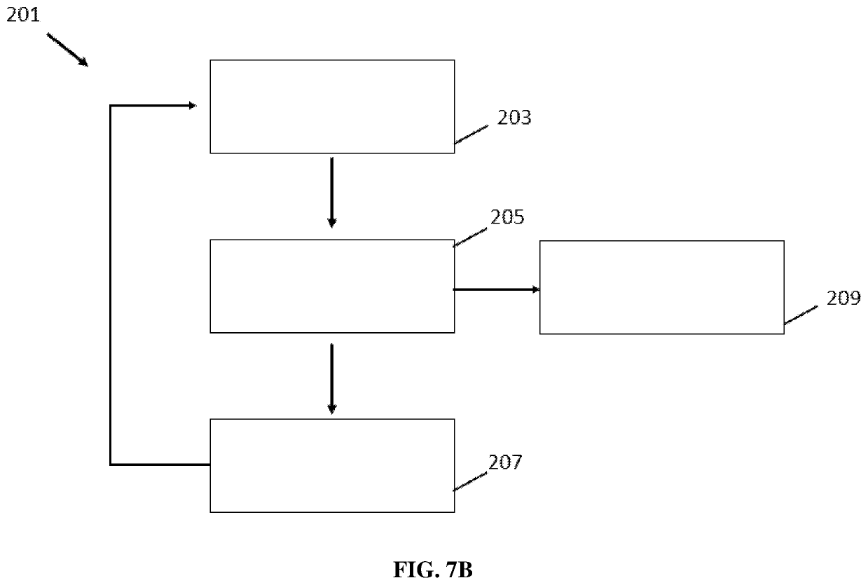

This example provides an example of a method 101 for detecting an replacing a sequence which may cause an undesired variant in a gene construct. As seen in FIG. 7A, the method 101 includes at least the following steps. The method 101 includes a step of performing an in-silico analysis of the gene construct to detect a presence of the sequence which may cause the undesired variant 103. The method 101 also includes a step of replacing the detected sequence which may cause the undesired variant with an alternative sequence 105. In step 105, the alternative sequence is derived via the use of synonymous codon substitution. The method 101 includes a step of measuring a frequency percentage of the undesired variant expressed by the gene construct 107. Step 107 includes performing an in-vivo analysis of one or more genes expressed by the gene construct by performing a RNA-sequencing analysis of an RNA product transcribed from the gene construct, where the frequency percentage of the undesired variant is determined at least in part by using a splice-aware aligner from the RNA-sequencing analysis. The method 101 also includes repeating the in-silico analysis and replacing steps 103, 105 if the frequency percentage of the undesired variant in the gene product from the in-vivo analysis 107 is greater than a predetermined value of acceptable frequency percentage of the undesired variant.

In the method 101, step 107 requires using at least 2 separate aligners. Also, the method 101 is preferably used for gene constructs which encode a chimeric antigen receptor.

In the method 101, the predetermined value of acceptable frequency percentage of the undesired variant is 0.1% if the undesired variant negatively impacts exportation of a chimeric antigen receptor to a cell surface, and the predetermined value of acceptable frequency percentage of the undesired variant is 0.01% if the undesired variant is associated with changes to a binding domain of a chimeric antigen receptor.

In the method 101, the repeating of the in-silico analysis and replacing steps 103, 105 is not performed if the undesired variant has been previously characterized as causing a negligible impact on the expression or function of the chimeric antigen receptor.

### Example 2

This example provides an example of a method 201 for creating a gene product used in cell therapy. As seen in FIG. 7B, the method 201 includes at least the following steps. The method 201 includes a step of performing an in-silico analysis on a gene construct encoding the gene product to identify and alter a sequence that may causes an undesired variant 203. The method 201 also includes the step of replacing the detected sequence which may cause the undesired variant with an alternative sequence 205. In step 205, the alternative sequence is derived by using synonymous codon substitution. The method 201 includes the step of measuring a frequency percentage of the undesired variant expressed by the gene construct 207. Step 207 includes performing an in-vivo analysis of one or more genes expressed by the gene construct by performing a RNA-sequencing analysis of an RNA product transcribed from the gene construct. In step 207, the frequency percentage of the undesired variant is determined at least in part by using a splice-aware aligner from the RNA-sequencing analysis. The method 201 includes repeating the in-silico and replacing steps 203, 205 to create a new gene construct if the frequency percentage of the undesired variant in the gene product from the in-vivo analysis is greater than a predetermined value of acceptable frequency percentage of the undesired variant. The method 201 also includes the step of measuring a frequency percentage of the undesired variant expressed by the new gene construct 209. Step 209 includes performing an in-vivo analysis of one or more genes expressed by the new gene construct by performing a RNA-sequencing analysis of an RNA product transcribed from the new gene construct, where the frequency percentage of the undesired variant is determined at least in part by using a splice-aware aligner from the RNA-sequencing analysis.

In the method 201, step 207 requires using at least 2 separate aligners. Also, the method 201 is preferably used for gene constructs which encode a chimeric antigen receptor.

In the method 201, the predetermined value of acceptable frequency percentage of the undesired variant is 0.1% if the undesired variant negatively impacts exportation of a chimeric antigen receptor to a cell surface, and the predetermined value of acceptable frequency percentage of the undesired variant is 0.01% if the undesired variant is associated with changes to a binding domain of a chimeric antigen receptor.

In the method 201, the repeating of the in-silico analysis and replacing steps 203, 205 is not performed if the undesired variant has been previously characterized as causing a negligible impact on the expression or function of the chimeric antigen receptor.

### Example 3

TABLE 1
Example software packages used in the RNA-
seq Profiling and Bioinformatics Analysis.

| Package | Version |
|---|---|
| FastQC | 0.11.7 |
| STAR | 2.7.3a |
| HISAT2 | 2.1.0, 2.0.1 |
| TopHat2 | 2.1.0 |
| Sambamba | 0.6.6 |
| BowTie2 | 2.2.6 |
| SAMtools | 1.6, 1.9, 1.10 |
| R | 3.6.2 |
| Digest | 0.6.25 |
| Integrated Genome Viewer (IGV) | 2.4.19 |
| bcl2fastq | 2.17 |

### Example 4

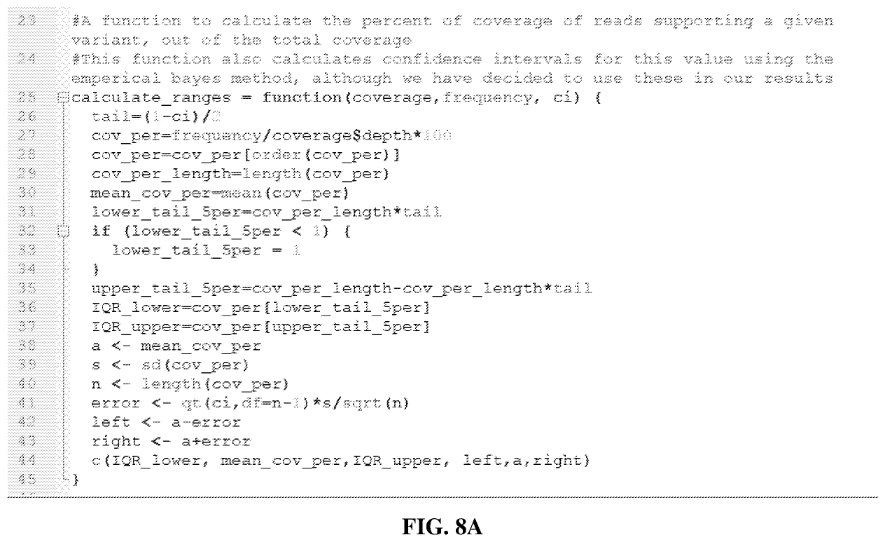

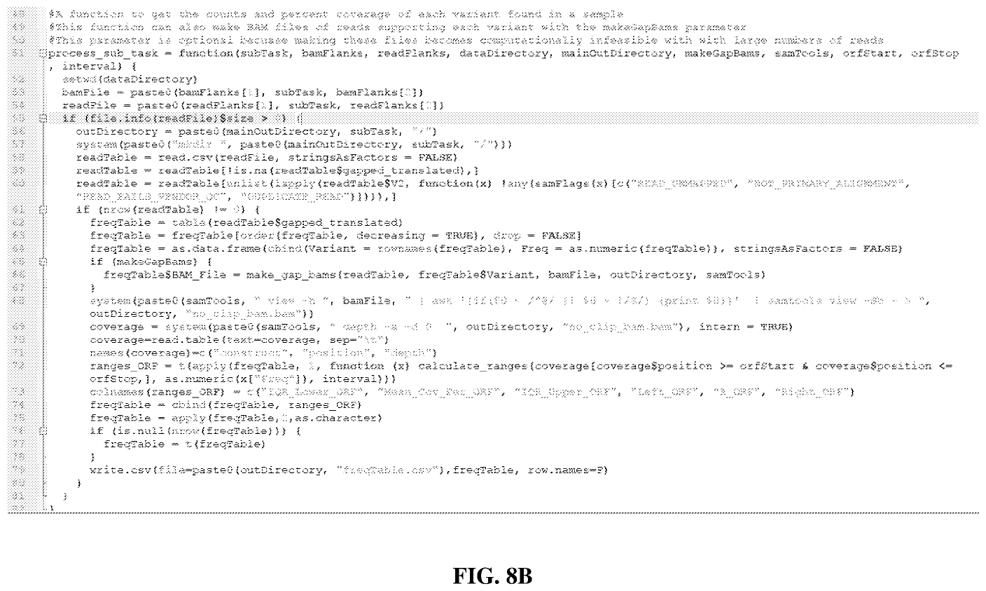

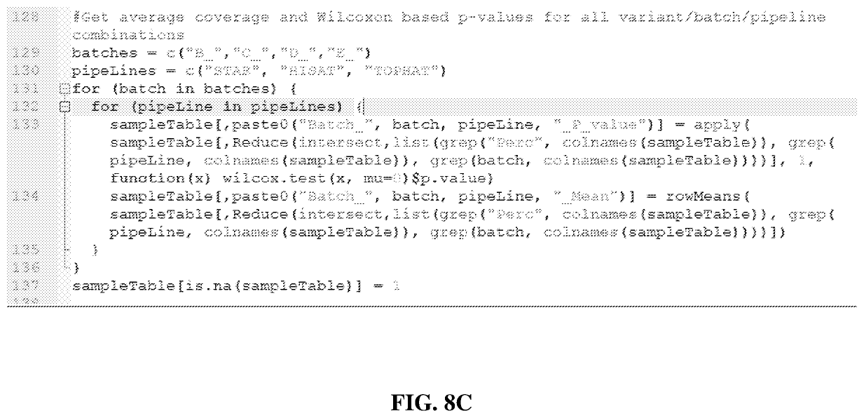

This example provides an example code portion that may be used to practice the disclosed methods. In the example code portion depicted in FIG. 8A, x is the percentage coverage, R is the number of reads supporting the gap, n is the number of bases in the open reading frame (ORF), and d is the read depth at each position in the ORF) (see app.R script lines 25-45).

### Example 5

This example provides an example code portion that may be used to practice the disclosed methods. In the example code portion depicted in FIG. 8B, the app.R script relies on SAMtools (version 1.10) depth function to calculate coverage for the construct and produce a BAM file (with calls to the SAMtools view function) with the reads from every unique gapped event for visualization purposes (see app.R 48-82).

### Example 6

This example provides an example code portion that may be used to practice the disclosed methods. In the example code portion depicted in FIG. 8C, OA conservative threshold for p-value <0.01 was selected to minimize false positives rate due to large number of spurious putative variants supported by <5 reads in a single donor and likely the result of sequencing artifacts. (see app.R script lines 131-136)

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments/aspects have been illustrated and described, it will be appreciated that various changes can be made without departing from the spirit and scope of the disclosure.

1. A method for detecting and replacing a sequence which may cause an undesired variant in a gene construct, comprising:
performing an in-silico analysis of the gene construct to detect a presence of the sequence which may cause the undesired variant;
replacing the detected sequence which may cause the undesired variant with an alternative sequence, wherein the alternative sequence is derived comprising synonymous codon substitution;
measuring a frequency percentage of the undesired variant expressed by the gene construct comprising performing an in-vivo analysis of one or more genes expressed by the gene construct comprising performing a RNA-sequencing analysis of an RNA product transcribed from the gene construct, wherein the frequency percentage of the undesired variant is determined at least in part by using a splice-aware aligner from the RNA-sequencing analysis; and
repeating the in-silico analysis and replacing steps if the frequency percentage of the undesired variant in the gene product from the in-vivo analysis is greater than a predetermined value of acceptable frequency percentage of the undesired variant.

2. The method of claim 1, wherein the gap-aware alignment comprises using at least two separate aligners.

3. The method of claim 1, wherein the in-silico analysis further comprises:
detecting at least one of a plurality of homologous sequences and a plurality of identical sequences within the gene construct, wherein the at least one of the plurality of homologous sequences and the plurality of identical sequences may cause an undesired variant in the gene construct; and
replacing any such detected plurality of homologous sequences and plurality of identical sequences comprising a step of synonymous codon substitution.

4. The method of claim 1, wherein the in-silico analysis further comprises calculating a matrix of subsection combinations from the gene construct and acquiring a Hamming distance for each of the subsection combinations.

5. The method of claim 1, wherein the in-silico analysis further comprises substituting a plurality of random synonymous codons in the gene construct with a plurality of alternative sequences such that plurality of alternative sequences increases a sum over the matrix.

6. The method of claim 1, wherein the gene construct comprises a sequence encoding a chimeric antigen receptor.

7. The method of claim 1, wherein the predetermined value of acceptable frequency percentage of undesired variant is determined based on whether the undesired variant is associated with at least one of whether the undesired variant negatively impacts exportation of the chimeric antigen receptor to a cell surface, whether the undesired variant is associated with changes to a binding domain of the chimeric antigen receptor, and whether the undesired variant has been previously characterized as causing a negligible impact on the expression or function of the chimeric antigen receptor.

8. The method of claim 1, wherein the predetermined value of acceptable frequency percentage of the undesired variant is 0.1% if the undesired variant negatively impacts exportation of the chimeric antigen receptor to a cell surface, and wherein the predetermined value of acceptable frequency percentage of the undesired variant is 0.01% if the undesired variant is associated with changes to a binding domain of the chimeric antigen receptor.

9. The method of claim 1, wherein the repeating the in-silico analysis and replacing steps is not performed if the undesired variant has been previously characterized as causing a negligible impact on the expression or function of the chimeric antigen receptor.

10. The method of claim 1, further comprising identifying and removing a subpopulation of high-frequency variants and identifying a subpopulation of low-frequency variants, and
wherein the in vivo analysis further comprises conducting an analysis to determine whether the subpopulation of low-frequency variants should be replaced.

11. A method for creating a gene product used in cell therapy, comprising:
performing an in-silico analysis on a gene construct encoding said gene product to identify and alter a sequence that may causes an undesired variant;
replacing the detected sequence which may cause the undesired variant with an alternative sequence, wherein the alternative sequence is derived comprising synonymous codon substitution;
measuring a frequency percentage of the undesired variant expressed by the gene construct comprising performing an in-vivo analysis of one or more genes expressed by the gene construct comprising performing a RNA-sequencing analysis of an RNA product transcribed from the gene construct, wherein the frequency percentage of the undesired variant is determined at least in part by using a splice-aware aligner from the RNA-sequencing analysis;
repeating the in-silico and replacing steps to create a new gene construct if the frequency percentage of the undesired variant in the gene product from the in-vivo analysis is greater than a predetermined value of acceptable frequency percentage of the undesired variant; and
measuring a frequency percentage of the undesired variant expressed by the new gene construct comprising performing an in-vivo analysis of one or more genes expressed by the new gene construct comprising performing a RNA-sequencing analysis of an RNA product transcribed from the new gene construct, wherein the frequency percentage of the undesired variant is determined at least in part by using a splice-aware aligner from the RNA-sequencing analysis.

12. The method of claim 11, wherein the gap-aware alignment comprises using at least two separate aligners.

13. The method of claim 11, wherein the in-silico analysis further comprises:
detecting at least one of a plurality of homologous sequences and a plurality of identical sequences within the gene construct, wherein the at least one of the plurality of homologous sequences and the plurality of identical sequences may cause an undesired variant in the gene construct; and
replacing any such detected plurality of homologous sequences and plurality of identical sequences comprising a step of synonymous codon substitution.

14. The method of claim 11, wherein the in-silico analysis further comprises calculating a matrix of subsection combinations from the gene construct and acquiring a Hamming distance for each of the subsection combinations.

15. The method of claim 11, wherein the in-silico analysis further comprises substituting a plurality of random synonymous codons in the gene construct with a plurality of alternative sequences such that plurality of alternative sequences increases a sum over the matrix.

16. The method of claim 11, wherein the gene construct comprises a sequence encoding a chimeric antigen receptor.

17. The method of claim 11, wherein the predetermined value of acceptable frequency percentage of undesired variant is determined based on whether the undesired variant is associated with at least one of whether the undesired variant negatively impacts exportation of the chimeric antigen receptor to a cell surface, whether the undesired variant is associated with changes to a binding domain of the chimeric antigen receptor, and whether the undesired variant has been previously characterized as causing a negligible impact on the expression or function of the chimeric antigen receptor.

18. The method of claim 11, wherein the predetermined value of acceptable frequency percentage of the undesired variant is 0.1% if the undesired variant negatively impacts exportation of the chimeric antigen receptor to a cell surface, and wherein the predetermined value of acceptable frequency percentage of the undesired variant is 0.01% if the undesired variant is associated with changes to a binding domain of the chimeric antigen receptor.

19. The method of claim 11, wherein the repeating the in-silico analysis and replacing steps is not performed if the undesired variant has been previously characterized as causing a negligible impact on the expression or function of the chimeric antigen receptor.

20. The method of claim 11, further comprising identifying and removing a subpopulation of high-frequency variants and identifying a subpopulation of low-frequency variants, and
wherein the in vivo analysis further comprises conducting an analysis to determine whether the subpopulation of low-frequency variants should be replaced.
